(19) United States
(12) Plant Patent Application Publication
(10) Pub. No.: US 2012/0284888 P1
(43) Pub. Date: Nov. 8, 2012

(54) DAHLIA PLANT NAMED 'DAATTEN'

(50) ;

(75) Inventor: Rune Harboe Nielsen, Groennegyden (DK)

(73) Assignee: Dalina ApS

(21) Appl. No.: 13/067,059

(22) Filed: May 5, 2011

(51) Int. Cl. A01H 5/00

(52) U.S. Cl. PLT/321

(57) ABSTRACT

A new distinct cultivar of Dahlia plant named ‘DAATTEN’, characterized by its compact growth with cylindrical upright to slightly spreading shape; stiff and strong lateral stems and thick single and compound leaves with up to 3 leaflets; color of mature leaves RHS 147A (upper side), RHS 191B (Underside); compound leaves 12-18 cm in length, and 12-15 cm in width and single leaves 6-13 cm in length and 6-9 cm in width; composite flower heads 11-12 cm in diameter with a large number of ray florets (about 120 to 140); and white ray florets (RHS 155B/155C) with small area of yellow near base (RHS 1B).

### LATIN NAME OF THE GENUS AND SPECIES OF THE CLAIMED PLANT

Dahlia Cav.

### VARIETY DENOMINATION

‘DAATTEN’

### BACKGROUND OF THE INVENTION

The present invention relates to a new and distinct cultivar of Dahlia plant, botanically known as Dahlia Cav., of the Compositae (Asteraceae) family, hereinafter referred to by the cultivar name ‘DAATTEN’.

The new Dahlia cultivar is a product of a planned breeding program conducted by the inventor, Rune Nielsen, in Stige, Denmark. The objective of the breeding program is to develop a new Dahlia cultivar with an upright, strong and healthy growth habit, suitable for large scale container production and with good garden performance; fully double flowers creating globular-shaped flower head (composite flower heads with many ray florets) with an attractive inflorescence color.

The new Dahlia cultivar originated from a cross, made in a controlled breeding program by the inventor in 2006, in Stige, Denmark. The female or seed parent is white Dahlia variety designated 4416B (not patented). The male or pollen parent is a yellow colored Dahlia variety designated 5218A (not patented).

Asexual reproduction of the new Dahlia cultivar by terminal cuttings was first performed in November, 2007 in Stige, Denmark, and has demonstrated that the combination of characteristics as herein disclosed for the new cultivar are firmly fixed and retained through successive generations of asexual reproduction. The new cultivar reproduces true to type.

### BRIEF SUMMARY OF THE INVENTION

The following traits have been repeatedly observed and are determined to be unique characteristics of ‘DAATTEN’, which in combination distinguish this Dahlia as a new and distinct cultivar:

1. Compact cultivar, with cylindrical upright to slightly spreading shape;

2. Compact cultivar with stiff and strong lateral stems and thick single and compound leaves, with up to 3 leaflets. Color of mature leaves yellow-green (RHS 147A) (upper side), greyed-green (RHS 191B) (Underside);

3. Compound leaves 12-18 cm in length, and 12-15 cm in width. Single leaves 6-13 cm in length and 6-9 cm in width;

4. Composite flower heads, 11-12 cm in diameter, with a large number of ray florets (about 120 to 140); and

5. White ray florets (RHS 155B/155C) with small area of yellow near base (RHS 1B).

Plants of the new Dahlia ‘DAATTEN’ differ from plants of the parents, 4416B (unpatented) and 5218A (unpatented) in the traits described in Table 1.

TABLE 1
Comparison with Parent Varieties

| | | Female Parent | Male Parent |
|---|---|---|---|
| | New Cultivar | 4416B | 5218A |
| Trait | ‘DAATTEN’ | (unpatented) | (unpatented) |
| Plant Size | | | |
| Height: | About 25-30 cm. | About 25-30 cm. | About 30-45 cm. |
| Diameter: | About 40-45 cm. | About 35-40 cm. | About 40-45 cm. |
| Overall Plant | Cylindrical, | Cylindrical, | Upright to |
| Shape: | upright, with | upright, with | spreading, with |
| | basal branching. | basal branching. | basal branching. |
| | Inflorescences in | Inflorescences in | Inflorescences in |
| | composite heads. | composite heads. | composite heads. |
| Stem | | | |
| Length: | 8-18 cm. | 8-16 cm. | 8-18 cm. |
| Diameter: | 8-12 mm | 8-12 mm | 8-12 mm |
| Strength: | Stiff and strong. | Stiff and strong. | Medium strong. |
| Color: | RHS 144A | RHS 144A | RHS 144A |
| | (yellow-green). | (yellow-green). | (yellow-green). |
| Number of | About 15. | About 12. | About 15. |
| Inflorescence | | | |
| heads per Plant: | | | |
| Flower | About 11-12 cm. | About 10 cm. | About 12 cm. |
| (Capitalum) | | | |
| Diameter: | | | |
| Color of ray | RHS 155B/155C | RHS 155B | Yellow cactus |
| florets | (white), RHS 1B | (white). | flower. |
| Upper side: | (green-yellow) | | |
| | near the base. | | |

Of the many commercial cultivars known to the present inventor, the most similar in comparison to the new Dahlia ‘DAATTEN’ is the Dahlia cultivar ‘Gallery Art Fair’, in the characteristics described in Table 2:

TABLE 2
Comparison with Comparison Variety

| | New Cultivar | Comparison Cultivar |
|---|---|---|
| Trait | ‘DAATTEN’ | ‘Gallery Art Fair’ |
| Plant Size | | |
| Height: | About 25-30 cm. | About 25-30 cm. |
| Diameter: | About 40-45 cm. | About 40-45 cm. |
| Overall Plant Shape: | Cylindrical, | Cylindrical, |
| | upright, with | upright, with |
| | basal branching. | basal branching. |
| | Inflorescences in | Inflorescences in |
| | composite heads. | composite heads. |
| Stem | | |
| Length: | 8-18 cm. | 8-18 cm. |
| Diameter: | 8-12 mm | 8-12 mm |
| Strength: | Stiff and strong. | Stiff and strong. |
| Color: | RHS 144A | RHS 144A |
| | (yellow-green). | (yellow-green). |
| Leaves: | | |
| Texture | Rugose. | Glabrous, shining. |
| Colour mature | RHS 147A | RHS N189A |
| upperside: | (yellow-green) | (greyed-green) |
| Number of Inflorescence | About 15. | About 10. |
| heads per Plant: | | |
| Bud color | RHS 144B | RHS 151C |
| | (yellow-green) | (yellow-green) |
| Flower (Capitalum) | About 11-12 cm. | About 12 cm. |
| Diameter: | | |
| Color of ray florets | RHS 155B/155C | RHS 155B/155C |
| Upper side: | (white), RHS 1B | (white), RHS 1B |
| | (green-yellow) | (green-yellow) |
| | near the base. | near the base. |

### BRIEF DESCRIPTION OF THE PHOTOGRAPHS

The patent or application file contains at least one drawing executed in color. Copies of this patent or patent application publication with color drawing(s) will be provided by the Office upon request and payment of the necessary fee.

The accompanying photographs illustrate the overall appearance of the new Dahlia ‘DAATTEN’ showing the colors as true as is reasonably possible with colored reproductions of this type. Colors in the photographs may differ slightly from the color value cited in the detailed botanical description, which accurately describe the color of ‘DAATTEN’.

FIG. 1 shows a close-up view of the composite flower head from ‘DAATTEN’.

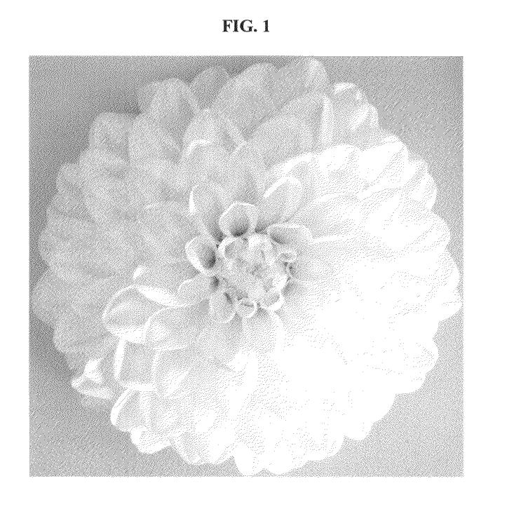

FIG. 2 shows a side view perspective of a typical flowering plant of ‘DAATTEN’ in a 19 cm pot, at 12 weeks of age after potting the young plant.

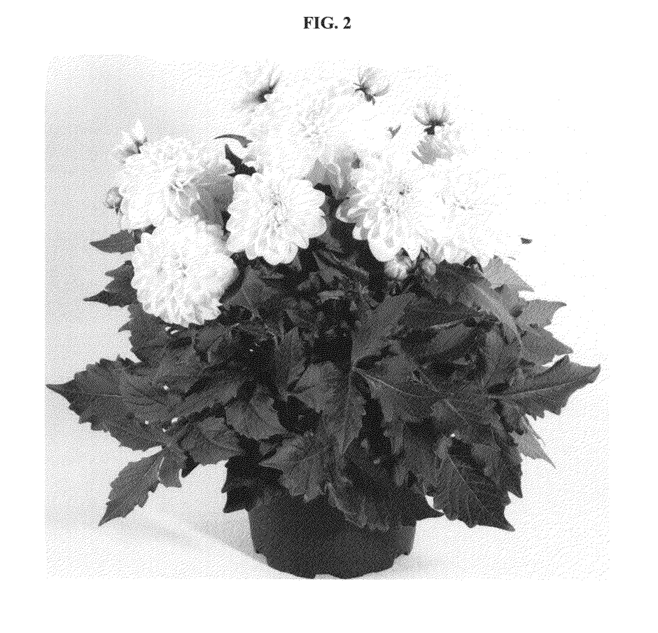

FIG. 3 shows a close-up view of different development stages of the composite flower head of ‘DAATTEN’.

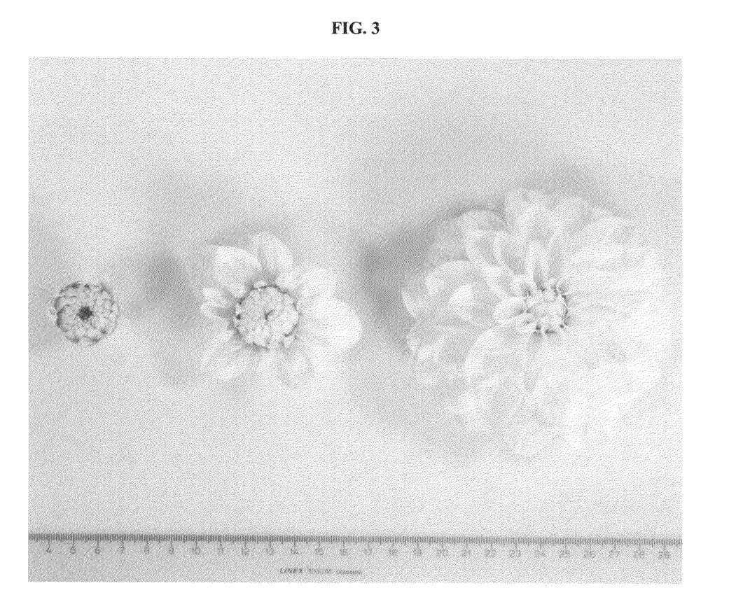

FIG. 4 shows a close-up view of the 120-140 ray florets from one composite flower head of ‘DAATTEN’

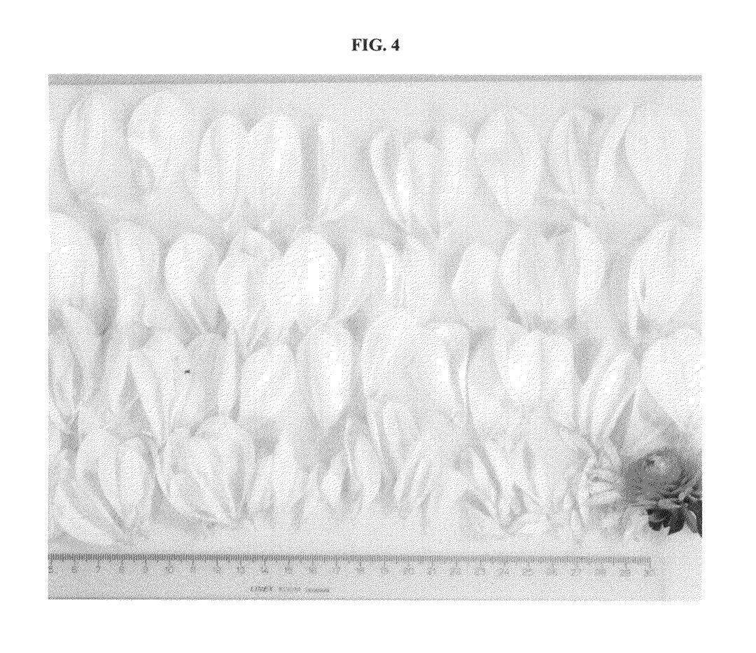

FIG. 5 shows a close-up view of the different single leaf sizes of ‘DAATTEN’.

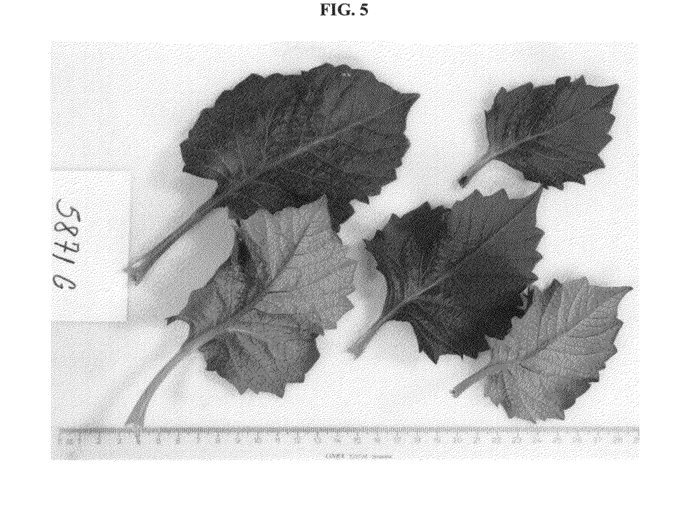

FIG. 6 shows a close-up view of the different compound leaf sizes of ‘DAATTEN’.

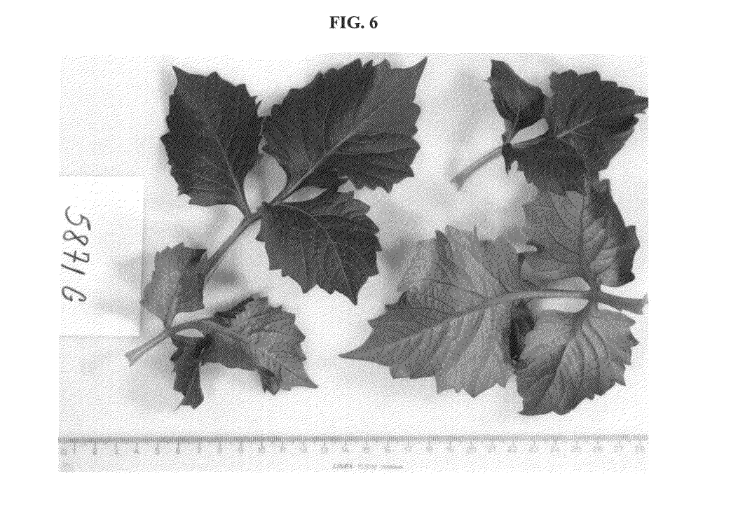

FIG. 7 shows a comparison between ‘Gallery Art Fair’ and ‘DAATTEN’, (Breeders reference: 5871G).

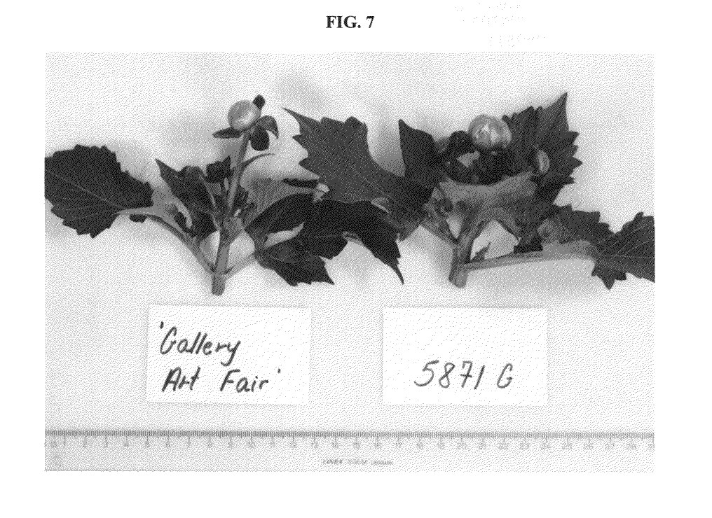

### DETAILED BOTANICAL DESCRIPTION

The new Dahlia ‘DAATTEN’ has not been observed under all possible environmental conditions. The phenotype of the new cultivar may vary with variations in environment such as temperature, light intensity, and day length without any change in the genotype of the plant.

The aforementioned photographs, together with the following observations, measurements and values describe the new Dahlia ‘DAATTEN’ as grown in a protected environment in a glass greenhouse in Fyn, Odense, Denmark, under conditions which closely approximate those generally used in commercial practice. During propagation, conducted in a glasshouse, vegetative cuttings were planted in small propagation pots with peat as substrate, and then placed in a plastic tunnel averaging about 21° C. and received photoperiodic treatments of 18 hours. Supplementary light was given when natural light fell below 3000-4000 Lux. Rooting occurred about 12 days after planting. In third week after planting the cutting, young plants were potted in an 19 cm pot in a glass covered greenhouse, maintained at 18 ° C. to 22° C. during the day, and at 17 ° C. to 19° C. during the night. Photoperiodic treatments were continued at 18 hours, and supplementary light was given when natural light fell below 3000-4000 Lux. Irrigation was done with water. The EC measured in the soil was maintained between 2.0 to 3.0. One week after potting, the first growth regulation was given: a spray with 85% daminozide, 0.2%, 40-100 ml/m2. During the production time, 9 additional sprayings were given with 85% daminozide, 0.2%, 40-100 ml/m2.

Color references are made to The Royal Horticultural Society Colour Chart (R.H.S.), 2001 edition, except where general colors of ordinary significance are used. Color values were taken under March daylight conditions at approximately 12:00 PM and 2:00 PM in Fyn, Odense, Denmark. The age of the ‘DAATTEN’ plants described is 15 weeks (including propagation time).
Classification:
   Botanical.—Dahlia Cav.
Parentage:
   Female or seed parent.—Dahlia variety designated 4416B (Unpatented).
   Male or pollen parent.—Dahlia variety designated 5218A (Unpatented).
Propagation:
   Type.—Vegetative terminate cuttings.
   Time and temperature to initiate roots.—About 9 to 12 days at 21° C. in tunnels or flat covered in a greenhouse.
   Time and temperature to produce a rooted young plant.—About 28 days at 21° C. during rooting phase, followed by 18° C.
   Rooting habit.—Fine, fibrous; Color of root: RHS 158A Yellow-white.
   Tuber development.—Plants might develop tubers in late fall when planted outside.
Plant description:
   General appearance and form.—Cylindrical, upright, with basal branching. Inflorescences in composite heads.
   Growth rate.—Growing about 4 to 6 cm per week during production period.
   Plant height (from soil level to top of plant plane).—About 25 cm to 30 cm.
   Plant width (spread).—About 40 cm to 45 cm.
   Crop time to produce a mature flowering plant.—It requires 3 to 4 weeks to produce a young plant in a 35 mm propagation plug. After potting 9 to 12 weeks are required to produce finished flowering plants in 19 cm pots.
Stem:
   Shape.—Round.
   Length.—About 8 cm to 18 cm.
   Diameter.—About 8 mm to 12 mm.
   Strength.—Stiff and strong.
   Aspect.—Upright to outward.
   Texture.—Smooth and glabrous, slightly furrowed.
   Color.—Mature: RHS 144A. Immature: RHS 144A.
Branches:
   Branching habit.—Basal branching with small lateral branches.
   Number of basal branches per plant.—About 3-5 (when pinched above 3 pair of leaves).
   Length (including flowers).—About 8 cm to 18 cm.
   Diameter.—About 8 mm to 12 mm.
   Strength.—Stiff and strong.
   Aspect.—Upright to spreading.
   Texture.—Smooth and glabrous.
   Color.—Mature: RHS 144A. Immature: RHS 144A.
   Internodes length.—About 15 to 20 mm.
   Internodes color.—RHS 144A.
Foliage description:
   Type.—Lower leaves: Single. Middle leaves: Compound, 3 lobed. Upper leaves: Single.
   Number of leaves per branch.—About 8 to 12 per branch.
   Color of leaves (leaves and leaflets).—Color of upper side (mature) RHS 147A Color of underside (mature) RHS 191B Color of upper side (immature) RHS 138A Color of underside (immature) 138A.
   Venation (leaves and leaflets).—Pattern: Pinnate Color of upper side: RHS 148A Color of underside: RHS 146A.
Compound leaves:
   Number of leaflets per compound leaf.13 3.
   Length of compound leaf.—12 cm to 18 cm.
   Width of compound leaf.—12 cm to 15 cm.
   Petiole of compound leaves.—Shape: Semi circular, with deep furrow on adaxial side. Length: 2.5 cm to 5 cm. Diameter: 3 mm to 6 mm. Texture: Smooth and glabrous. Color: RHS 146B.
   Leaflets of compound leaf.—leaflet quantity: 1 Terminal leaflet length: 8 cm to 13 cm. Terminal leaflet width: 5 cm to 8 cm. Terminal leaflet shape: Ovate. Terminal leaflet shape at apex: Acuminate. Terminal leaflet shape at base: Cordate to rounded. Terminal leaflet margin: Dentate. Terminal leaflet texture (both sides): Rugose. Rachis length: 1 cm to 2 cm. Rachis Diameter: 3 mm to 4 mm. Rachis color: RHS 146B. Lateral leaflet quantity: 2 Lateral leaflet length: 6 cm to 8 cm. Lateral leaflet width: 4 cm to 6 cm. Lateral leaflet overall shape: Ovate. Lateral leaflet shape at apex: Acuminate (asymmetric). Lateral leaflet shape at base: Rounded. Lateral leaflet margin: Dentate. Lateral leaflet texture (both sides): Rugose.
Simple leaves: Simple leaves overall shape: Ovate. Simple leaves shape at apex: Acuminate. Simple leaves shape at base: Cordate to rounded. Simple leaves length: 6 cm to 13 cm. Simple leaves width: 6 cm to 9 cm. Simple leaves margin: Dentate. Simple leaves texture: Rugose. Petiole shape: Semi circular, with deep furrow on adaxial side. Petiole length: 2.5 cm to 5 cm. Petiole diameter: 3 mm to 6 mm. Petiole texture: Smooth and glabrous. Petiole color: RHS 146B.
Inflorescence description:
   Natural flowering season.—Grown outside as a bedding plant, flowering occurs continuously during growing season from spring to autumn (In Denmark, from June to beginning of October). Plants can be brought to flower anytime when grown under the recommended greenhouse conditions.
   Time to flower.—60 to 80 days from potting a young plant until flowering.
   Inflorescence longevity on the plant.—Inflorescence will maintain good color and substance for about 6-10 days; however, the longevity of individual inflorescence is highly dependent on temperature and light conditions. Inflorescence persistent.
   Type.—Composite flower heads.
   Arrangement and shape.—Persistent, single, composite inflorescences from leaf axils. Disc and ray florets arranged acropetally in a composite flower head. Upright, slightly spreading. Terminal young flower head/buds initially 0-20°. Buds from axillary shoots 1-60°. When flower opens 0-30°.
   Quantity of inflorescences heads.—Freely flowering; about 15 buds and open inflorescences per plant.
   Fragrance.—None.
   Bud (inflorescences head).—
   Rate of opening (from showing color to fully open).—4 to 10 days. The rate of opening is highly dependent on temperature and light conditions. Length: About 12-14 mm. Diameter: About 16-22 mm. Shape: Flat globular. Texture: Glabrous, shining. Color RHS 144A developing into RHS 144B.
   Peduncle.—From both terminate and axillary shoots. Length: About 4-12 cm. Diameter: About 3-5 mm. Angle: About 0-20° from vertical. Strength: Stiff and strong. Texture: Glabrous. Color: RHS 144A.
   Inflorescence head.—Depth (height): 6-7 cm. Diameter: 11-12 cm.
   Ray florets.—Arrangement: Imbricate, in about 9 to 14 whorls of ray florets, each with 9 to 11 florets to equal a total of about 120-140 ray florets per flower head, depending on light and temperature conditions. Quantity per Inflorescence head: 120-140. Length: 50 mm. Widths: 22-25 mm. Overall shape: Obovate Apex shape: Obtuse Base shape: Acuminate, slightly fused. Margin: Entire Texture: Glabrous (upper side and underside). Colour when opening fused ray floret starting to show RHS color: 160D. Colour when fully opened Color upper side: RHS 155B/155C with RHS 1B at small area near the base. Color underside: RHS 155C.
   Disc florets.—Arrangement: 30-40 yellow-orange disc florets arranged in center of inflorescence head. Apperance: Quantity per Inflorescence head: 30-40. Length: 8-10 mm. Diameter: 2 mm. Overall shape: Tubular. Apex shape: Star with 5 triangular tips. Base shape: Fused to tube. Color RHS 17A, RHS 145C near base.
   Phyllary.—Arrangement: One subtending each floret. Quantity: 120-140. Length: 12-22 mm. Width: 4-6 mm. Overall shape: Rounded. Shape at apix: Acute with rounded tip. Shape at base: Fused. Margin: Entire. Color: Mature: RHS 151B; RHS 146A on underside of the 8-10 phyllaries in the outer whorl Immature: RHS 151B.
   Bracts.—Arrangement and appearance: Reflexed, involucral bracts. Quantity: 5. Length: 13-16 mm. Width: 6-8 mm. Overall shape: Subulate. Apex shape: Acute. Base shape: Sessile. Margin: Entire Texture: Glabrous. Colour mature Upper side: RHS 137A Underside: RHS 137A Colour immature Upper side: RHS 137A Underside: RHS 137A.
Reproductive organs:
   Androecium.—Location: Disc florets only. Stamen number: 5, fused into synandrous tube around style. Stamen length: About 6-10 mm. Anther shape: Tubular. Anther length: About 5-6 mm. Pollen amount: Medium amount. Pollen color: RHS 21A.
   Gynoecium.—Location: Ray and disc florets. Quantity: 1. Pistil length: 6-10 mm. Stigma shape: Bifurcate. Stigma length: About 2 mm. Stigma color: RHS 151C Style length: About 4 mm. Style color: RHS 151C. Ovary color: RHS 151C.
Seed/fruit: Seed and fruit production has not been observed.
Disease/pest resistance: Resistance to pathogens and pests common to Dahlia has not been observed.
Disease/pest susceptibility: Susceptibility to pathogens and pests common to Dahlia has not been observed.
Temperature tolerance: Plants of the new Dahlia have exhibited good tolerance to rain, wind and drought; however, flowering may cease during hot periods (temperatures above 25° C.). Low temperature tolerance to 1° C.
Growth regulators: Daminozide (85% water soluble dry concentrate formulation).

What is claimed is:

1. A new and distinct cultivar of Dahlia plant named ‘DAATTEN’, as illustrated and described herein.
